(19) United States
(12) Patent Application Publication
(10) Pub. No.: US 2005/0266547 A1
(43) Pub. Date: Dec. 1, 2005

(54) Cell culture chamber for a cell culture system

(76) Inventors: Wilhelm Scherze, Spargelweg (DE); Josef Seidl, Matthias (DE)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/528,058

(22) PCT Filed: Sep. 16, 2002
(86) PCT No.: PCT/EP02/10359

§ 371 Date: Mar. 15, 2005

(51) Int. Cl. C12M 1/34

(52) U.S. Cl. 435/287.1

(57) ABSTRACT

A cell culture chamber (20) for a closed cell culture system serving to continuously supply different cells with liquid nutrient media, growth factors, gases or the like essentially consists of a membrane plate (1) with a membrane (2) for accommodating at least one cell culture and with a number of channels (4, 4′, 4″, 4′″) for supplying liquid, gassing and connecting sensors, of a transparent glass pane (3), which is placed on one side of the membrane plate (1) while provided for observing the inside of the cell culture chamber (20) from this side, and consists of a connecting plate (5), which is placed on the opposite side of the membrane plate (1) and which is provided with an incorporated, transparent glass pane (6) for illuminating the inside of the cell culture chamber (20) from this opposite side with the aid of an assigned lighting system.

The invention relates to a cell culture chamber for a closed cell culture system serving to continuously supply cells of the most diverse type with liquid nutrient media, growth factors, gases and the like.

Essentially cell culture means a culture which is prepared on the basis of individual cells that either result from parts of tissue, primary cultures, from cell lines or cell stems obtained by enzymatic, mechanic or chemical disintegration. For the cultivation of the cells normally culture vessels made of plastic that are incubated in CO₂ incubators are used. This guarantees a constant temperature (e.g. 37° C.) and a buffering of the medium by means of a 5 to 10 percent CO₂ gassing. Oxygen supply is effected by simple diffusion. With the known equipment, co-cultivation and freely changeable incubation conditions are normally not possible.

For microscopic observation or for specific examinations, the culture vessels have to be taken out of the relevant incubators, whereby incubation is interrupted, the cells cool down and the test conditions are no longer constant as a result.

The previously known equipment or systems for the cultivation of cells, however, do no longer meet the requirements of modern cell culture technology.

With regard to current main research areas in the pharmaceutical industry in particular, these being the fields of inflammation (rheumatics), the fight against cancer, cardiovascular diseases, Aids, apoptosis (programmed cell death) and blood coagulation, the development and the testing of suitable new active agents and drugs by means of a cell culture system, which shall be designed in such way that enables the testing of substances and actions under almost in-vivo conditions, that means with an almost perfect mapping of complex, biological systems before passing on to the clinical phases (tests on test persons) is indispensable.

Out of consideration for the above-mentioned situation, there is demand for a method for simulating the progress of reactions within one or several organ system(s) (e.g. by means of a series connection of cell culture chambers with hepatocytes and other types of cells, testing for degradation products and metabolites) in order to considerably minimize, on the one hand, the period of time that passes between the identification of the action of a substance and drug approval and to enable the obtaining of necessary findings on the mechanism of action of the substance within a complex biological system before passing on to the clinical test phase, on the other hand.

A similar situation is given, for example, in the area of cosmetics industry as well.

State-of-the-art technology includes, for example, multivalent cell culture systems (see DE 199 15 178 A1, for example), problem-adapted cell culture systems for specific tasks (see WO 98/17822, for example) or methods for the replication of cell cultures (see WO 97/37001 for example).

Furthermore it is known from WO 99/23206 that there is a method for mixing a cell culture infected with varicella in cylindrical bottles, for example.

Finally a method and a device used to take up a cell culture are known from EP 0 999 266 A1; these aim at creating the most homogenous possible conditions for the molecular biological or genetic examination of cells.

Out of consideration for the situation in the field of modern cell culture technology described in the beginning, the target of the present invention is now to create a new cell culture chamber for a closed cell culture system serving to accommodate at least one cell culture, and the continuously supply in particular of different cells with liquid nutrient media, growth factors, gases and the like in the cell culture chamber is guaranteed, without cells of a culture having to be taken out of their habitual environment, while all cell cultures can be permanently examined under the microscope without the gassing having to be interrupted.

This task is solved with regard to a cell culture chamber for a closed cell culture system for the continuous supply of different cells with liquid nutrient media, growth factors, gases and the like according to the invention in such way that essentially the cell culture chamber is composed of the following components:
a) a membrane plate with a membrane for accommodation of a least one cell culture and with a number of channels for supplying liquid, gassing and connecting sensors;
b) a transparent glass pane, which is placed on one side of the membrane plate while provided for observing the inside of the cell culture chamber from this side; and
c) a cover plate, which is placed on the other opposite side of the membrane plate, which is provided with an incorporated, transparent glass pane for illuminating the inside of the cell culture chamber from the mentioned other side by means of an assigned lighting system.

Preferably here the transparent glass pane on the membrane plate for observing the inside of the cell culture chamber is fixed in the area of the underside of the membrane plate.

Even more preferably the cover plate forms a cell culture chamber cap with a fixed integrated transparent glass pane and the cell culture chamber cap is fixed on the upper side of the membrane plate in a releasable way.

According to a further embodiment of the invention, it is provided that the cell culture chamber cap as well as the underside of the membrane plate have a opening for the accommodation and fixation of the respective glass pane, in particular for fixation, which is not releasable.

Preferentially the respective transparent glass pane is a sapphire glass pane.

According to a further embodiment of the invention it may further more be provided that for the fixation of the membrane on the membrane plate a retaining ring is placed, which with the aid of the cell culture chamber cap can be pressed on the periphery of the membrane so that the latter can be fixed.

A further embodiment of the invention is that preferentially on the side of the cell culture chamber cap facing the membrane plate a joint ring is provided, by which, when the cell culture chamber is closed, the cell culture prepared on the membrane is aseptically closed.

Another, preferential development of the invention consists of the fact that by an suitable compartmentalization of the cell culture chamber a constant, continuous gassing is enabled via the respective assigned channels with freely selectable concentrations of the most different types of gases. In particular this has the advantage that the cell culture in the inside of the cell culture chamber can be observed without interrupting the gassing.

In addition, it is also possible that the membrane plate on its side opposite the cell culture chamber cap can be fixed on an assigned retainer plate for introduction into the cell culture system and this retainer plate is fitted with an integrated heating for the cell culture chamber. Preferentially this heating is an electrical heating.

If a direct co-cultivation shall be executed, it is especially advantageous to use a gas-permeable biofoil as membrane, as explained in more detail below.

Now in the following the invention will be explained in more detail by application examples and the following will be showed:

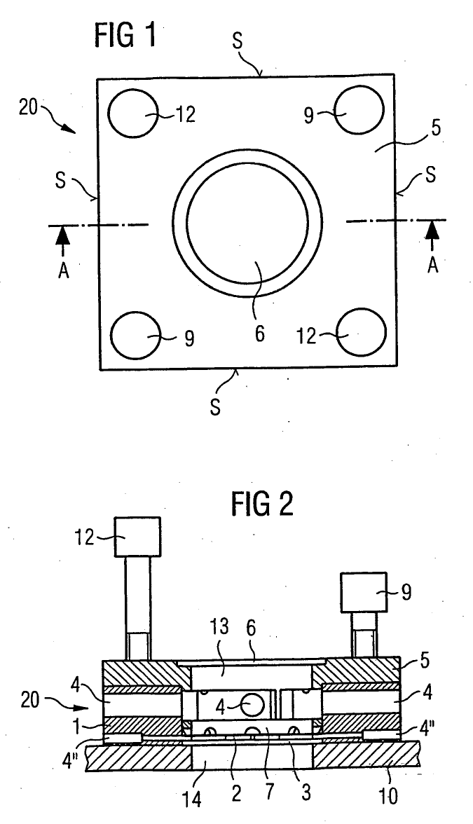

FIG. 1 shows by means of a diagram a top view of a cell culture chamber;

FIG. 2 shows a sectional view of a cell culture chamber according to line A-A in FIG. 1;

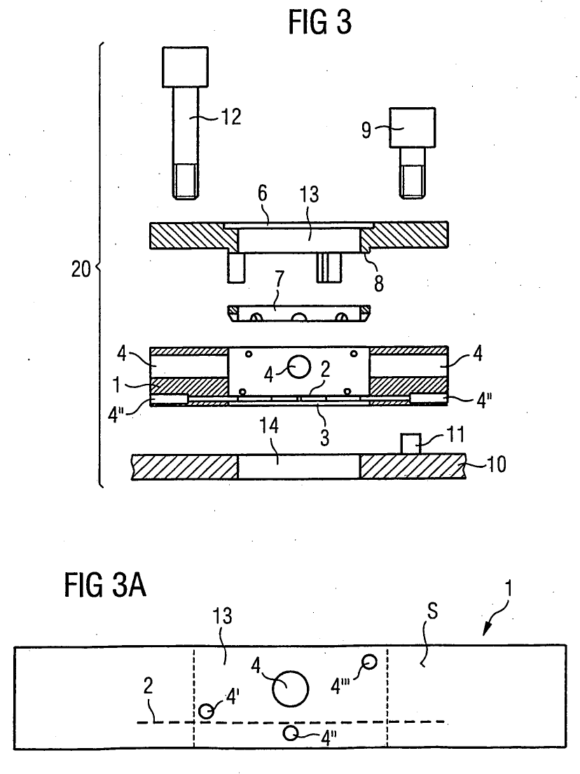

FIG. 3 shows the sectional view of the cell culture chamber according to FIG. 2 in an exploded view;

FIG. 3A shows, by means of a diagram, a side elevation of a membrane plate of the cell culture chamber; and

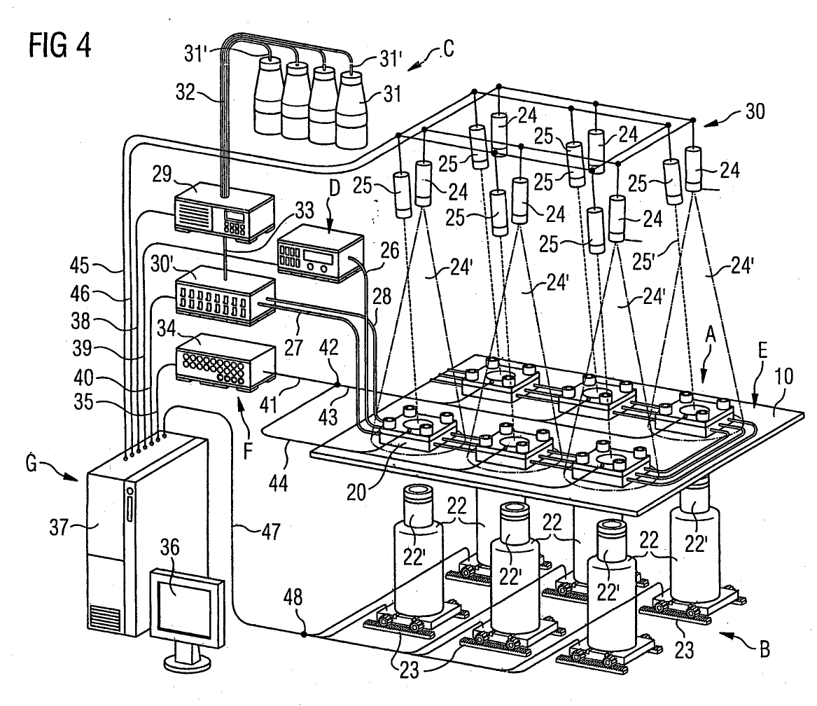

FIG. 4 shows by means of a diagram a complete, closed cell culture system, in which a predefined number of cell culture chambers is used.

According to the FIGS. 1, 2, 3 and 3A, a cell culture chamber 20 is materially composed of a membrane plate 1, in which a membrane 2, in particular a gas-permeable biofoil is placed, which serves to the accommodation of at least one cell culture. In the shown application engineering, the membrane 2 is placed in the lower area of the membrane plate 1, in particular securely fixed.

The membrane plate 1 also is fitted with a number of channels 4, 4′, 4″ and 4′″, which run in the inside of the cell culture chamber 20, and of which channel 4 serves to connect sensors, channel 4′ to supply liquids and gas, channel 4″ to supply liquids and channel 4′″ to withdraw liquids or gas, as shown in more detail below in FIG. 3A. Thus the cell culture can be equally supplied from above as well as from below. By the special system of channels in the membrane plate 1 it is in particular guaranteed that the required conditions of incubation can be realized.

In the area of the underside of the membrane plate 1, a transparent glass pane 3 is placed for observing the inside of the cell culture chamber 20. Such observation is preferentially carried out from the underside of the membrane plate 1 with the aid of a video camera with microscope adapter, as further explained below.

On the upper side of the membrane plate 1, a cell culture chamber cap 5 is placed, which forms an upper cover plate and is integrated in a transparent glass pane 6 for a lighting of the inside the cell culture chamber 20. The cell culture chamber cap 5 is fixed in particular on the upper side of the membrane plate 1 and, preferentially with the aid of screws 9, is bolted to the membrane plate 1 in a releasable way.

The cell culture chamber cap 5 as well as also the underside of the membrane plate 1 are provided with an opening for accommodating and fixing the respective glass pane 6 resp. 3.

On the cell culture chamber cap 5, the glass pane 6 preferentially covers a round opening 13. In an appropriate way at the membrane plate 1 the glass pane 3 forms a lower cover under membrane 2.

Preferentially the transparent glass panes 3 and 6 are each sapphire glass panes.

For fixation of the membrane 2 on the membrane plate 1, a retaining ring 7 is provided, which—with the aid of the cell culture chamber cap 5—can be pressed on the periphery of the membrane 2 in order to fix the latter in the cell culture chamber 20.

Further more a joint ring 8 is placed on the side of the cell culture chamber cap 5 facing the membrane plate 1. By means of this joint ring 8, the cell culture prepared on the membrane 2 is aseptically closed when the cell culture chamber 20 is closed (see FIG. 2).

FIG. 3A shows, by means of a diagram, a side view of the membrane plate 1 with the orifices of channel 4 serving to connecting sensors, channel 4′ serving to supply liquid or gas, channel 4″ serving to supply liquid and channel 4′″ serving to withdraw liquid or gas provided there.

The orifices of the channels 4, 4′, 4″, 4′″ as shown in FIG. 3A for one side S of the membrane plate 1, are identically provided on all three other sides of the membrane plate 1.

FIG. 3A also shows the placement of the gas-permeable membrane 2 in the inside of the membrane plate 1 with the placement of the membrane 2 being made in such a manner that a defined compartmentalization of the cell culture chamber 20 results from this, which enables a direct co-cultivation of two cell cultures. At such a direct co-cultivation on both sides of the membrane 2 a cell culture each of different type is placed and in particular the cells of the first cell culture growing on the one side of the membrane 2, i.e. on the apical side, are supplied with a first flow of media via channel 4′, whereas the cells of the second cell culture growing on the other side of the membrane 2, i.e. on the basolateral side, are supplied with a flow of media, which differ from the first one, via channel 4″. Thus the cells on the apical side act as cover layer, while the cells on the basolateral side act as internal cells.

Channel 4′ leading to the apical side may also serve to gassing, in particular to a constant, continuous gassing with freely selectable concentrations of the most different gases.

As already mentioned, channel 4 serves to connect sensors.

Finally channel 4′″ serves to withdraw liquids or gases from the apical side of the membrane 2.

The components of the cell culture chamber 20 particularly are made from appropriate stainless steel, for example from stainless steel 1.4435.

After fitting the membrane plate 1 in the clean room, the cell culture chamber cap is placed and bolted with the membrane plate 1 by means of the screws 9. The screws are short screws 9, which fix the cell culture chamber cap 5 on the membrane plate 1. Here the cell culture, which is accommodated by the membrane plate 1, is simultaneously closed in an aseptic way with the aid of the joint ring.

In this condition, the cell culture chamber 20 is assembled with a retainer plate 10 of a cell culture system (see FIG. 4).

For this purpose in particular, the membrane plate 1 on its side opposite to the cell culture chamber cap 5 is fixed on the retaining plate 10, which has a retainer bolt for adjustment. For the purpose of fixing the membrane plate 1 on the retaining plate 10, relatively long screws 12 are provided. Furthermore the retaining plate has an integrated heating, preferably an electrical heating for the cell culture chamber 20, as explained in more detail below.

In the shown application example, the cell culture chamber 20 is materially designed in a rectangular, parallelepiped form and has a square elevation. Naturally also other geometrical realizations are conceivable.

In the application example shown by means of the FIGS. 1-3A, the orifices of the channels 4, 4′, 4″ and 4′″ are provided on all four sides S of the membrane plate 1 in identical way. But also here other arrangements for these channels, which materially have a cylindrical form, are conceivable. Other cross sections of the channels are also conceivable.

It has to be mentioned, however, that the retaining plate 10 shows, for each cell culture chamber to be fixed on it, a medium circular opening 14, the cross section of which corresponds to the opening 13 of the cell culture chamber cap 5 opposite. This middle opening 14 of the retaining plate 10 ensures the observation of the inside of the cell culture chamber 20 from below by means of a video camera with a microscope adapter, as explained in more detail in FIG. 4.

FIG. 4 shows the application of the cell culture chambers 20 according to the invention in a closed cell culture system 30.

In this cell culture system 30 for example six cell culture chambers 20 are placed as group A on the retaining plate 10, which by its integrated heating E guarantees for the incubation during the operating time of the cell culture system 30 constant temperatures within each of the cell culture chambers 20 of the cell culture chamber group A.

In particular by means of this heating E, an electrical heating of the respective cell culture chamber 20 is effected, by which a very accurate temperature control is possible. In particular the heating E is designed in such a way that each individual cell culture chamber 20 of the cell culture chamber group A can be individually heated.

It is a special advantage of the cell culture system 30 that the heating E can be controlled by means of an assigned software. For this purpose a system consisting of infrared temperature measuring device 25 is installed above the cell culture chamber group A in such a way that a respective infrared temperature measuring device 25 is assigned to each individual cell culture chamber 20. The respective infrared temperature measuring device 25 senses, by means of a infrared beam 25′, the temperature prevailing in the cell culture and permanently signals the respective measurement result to a computer-controlled monitoring and control system G, which materially consists of a data processing system 37 and a monitor 36. The individual infrared temperature measuring device, 25 are connected to the monitoring and control system G via a joint interconnection line 45. If the initially preset temperatures in the cell culture chambers 20 of the cell culture chamber group A change, a control and/or adjustment of the heating E is automatically effected by the monitoring and control system G, i.e. the temperature prevailing in the individual cell culture chamber 20 is permanently adjusted to achieve a constant temperature. Instead of by means of infrared temperature measuring devices, the temperature control might also be effected by means of other suitable temperature sensors.

By means of the software included in the monitoring and control system G, on the other hand, it can be enabled that the temperatures in the individual cell culture chambers 20 of the cell culture chamber group A are freely adjustable and changeable over the entire duration of the experiment, if this should be necessary due to certain reasons.

For the purpose of a permanent microscopic observation of the inside of the relevant cell culture chamber 20, a video system B with an accordingly assigned microscope system is provided. This video system B will be explained in more detail in the following.

Below every individual cell culture chamber 20 of the cell culture chamber group A that includes a total of six cell culture chambers in this application example, a video camera 22 with a microscope adapter 22′ is fitted on a mechanically adjustable, mobile table, therefore there is a total of six video cameras 22 with accessory microscope adapters 22′. Thus one video camera 22 each with a microscope adapter 22′ serves to observe one cell culture chamber 20 each. After the experiment has been started and after meaningful areas in the cell culture contained in the relevant cell culture chamber 20 have been identified, an observation sector in the cell culture chamber 20 is determined. The mechanically adjustable mobile table 23 is moved to this observation sector then by means of adjusting screws (not represented), then the mobile table 23 is locked and the video system B remains in the same position over the entire duration of the experiment as a result. Furthermore the definition setting at the relevant microscope adapter 22′ is adjusted at the start of the test. This adjustment process on the relevant microscope adapter 22′ is carried out for all six cell culture chambers 20 and then remains unchanged until the experiment has been completed.

The video system B is preferably controlled via the software contained in the monitoring and control system G as well. Here every individual video camera 22 with microscope adapter 22′ is controlled in the process. This is carried out in particular in such a way that pictures of the relevant cell culture in the cell culture chamber 20 are taken at freely selectable intervals (every minute, for example), a light source 24 fitted above the relevant cell culture chamber 20 illuminating the relevant cell culture at the relevant point in time at which such a recording is made, so that a sufficient illumination of the inside of the cell culture chamber 20 is ensured for the video recordings. When the video recording is completed, the control switches the relevant light source 24 into a weak, dimmed-out standby state until the next video recording is made. The light beam and/or light cone that is emitted by each of the light sources 24 and that enters inside the relevant cell culture chamber 20 through the respective sapphire glass pane 6 is marked 24′ in FIG. 1.

All light sources 24 are connected to the monitoring and control system G via a joint connecting line 46.

Every single light beam/light cone 24′ illuminates the entire area of the cell culture contained in the relevant cell culture chamber 20.

The video system B is also connected to the monitoring and control system G via a line 47; from the monitoring system, the line 47 is connected to a junction point 48 to which the individual video cameras 22 are connected via correspondingly assigned lines.

The video system B with microscope system as described above is only one the possible models. Another possible embodiment of such a system for the permanent observation of the inside of the cell culture chambers comprises a single observation system, consisting of a video camera and a microscope adapter, is installed on a mobile table; this mobile table moves to the six cell culture chambers 20 of the cell culture chamber group A at freely selectable intervals. The adjustment of the observation system is carried out for the individual cell culture at the start of the test, this means preferably after meaningful areas have been identified in the relevant cell culture, by means of the respective software included in the monitoring and control system G, this means that the six target positions of the moving table on which the observation system has been mounted are programmed by means of the respective computer program. On account of the mechanic tolerances of the moving table, however, it is necessary to include an area that is larger than the area inside the individual cell culture chamber to be observed.

The software now serves to define the area to be observed within this larger area. The software is able to record and to recognize contours, this means that the contours and the configuration of the cells is recognized when the table moves in the direction of a cell culture chamber again and an initially defined observation area is recorded.

This observation system that has been explained last is not represented in detail in the drawings, but the individual cell culture chambers 20 are also illuminated by means of the light sources 24, as it has already been explained in details above.

Furthermore the cell culture system 30 represented in FIG. 4 is equipped with a dosage system C for liquids (e.g. liquid nutrient media and the like) that is fitted with e.g. four liquid storage tanks 31 with one assigned liquid take-off line 31′ each; these liquid take-off lines 31′ constitute a group of lines 32. This group of lines 32 is, on the other hand, connected to a pump system 29 through which the different cell culture chambers 20 of the cell culture chamber group A are supplied with freely selectable liquids that are contained in the liquid tanks 31.

The pump system 29, on the other hand, is connected to a multi-valve module 30′ via a line 33. The liquids are supplied to the cell culture chamber group A from the multi-valve module 30′ via sterile hose line systems 27 and 28; these liquids are passed on from the individual cell culture chambers 20 in a flexible manner. The liquid supply as well as the withdrawal and passing on of liquids is carried out via sterile hose systems that are installed with standard hose connecting elements and distributors at the start of a test; this means that they are connected to corresponding channels in the membrane plate 1 of a relevant cell culture chamber 20. Here the connection of the standard hose elements (not represented in detail in the drawings) with the assigned channels of the membrane plate is adjusted in such a way that sterility is guaranteed.

For reasons of flexibility, the types of liquids, the directions of flow, the distribution of liquids and their flow volumes can be changed and/or controlled during an experiment; this is preferably controlled by the computer-controlled monitoring and control system G. For this purpose, the pump system 29 is connected to the monitoring and control system G via a connecting line 38 and the multi-valve module 30′ via a connecting line 40.

Therefore the dosage system C of the cell culture system 30 enables you to supply the cell culture chamber group A with a variety of different liquids.

Furthermore the cell culture system 30 is equipped with a gassing system D for a variety of different gases. This gassing system D also serves to gas the different cell culture chambers 20 of the cell culture chamber group A with a variety of different gases, e.g. air, O₂, N₂, CO₂. From the gassing system D, the gas is supplied to the cell culture chamber group A via a sterile hose line 26. Also here the gases can be passed on from the different cell culture chambers 20 by means of the respectively assigned channels 4′ and 4′″ (see FIG. 3A) in a flexible manner.

The gas is altogether supplied withdrawn and passed on via sterile tubes that are installed by means of standard hose connecting elements and distributors at the start of an experiment.

The connections of the hose connecting elements with the correspondingly assigned channels 4′ and 4′″ of the membrane plate 1 are adjusted in such a way so that sterility is ensured.

With the gassing system D as well, the types of gases, the directions of flow, the gas distribution as well as the gassing concentration can be changed and/or controlled during an experiment for reasons of flexibility. For this purpose, the gassing system D, on the other hand, is connected to the monitoring and control system G that contains the relevant software for controlling the gassing system D via a connecting line 39.

Finally the cell culture system 30 further includes a monitoring system F with predefined sensor modules 34. By means of this monitoring system F, the relevant parameters in the relevant cell culture chamber 20 of the cell culture chamber group A can be measured, measured permanently in particular, using accordingly assigned sensors, for the entire duration of a test, these parameters being, for example, pH value, glucose, lactate, oxygen, electric potential, etc. For this purpose, the monitoring system F is connected to the individual cell culture chambers 20 of the cell culture chamber group A of the cell culture system 30 via a line 41, via a junction point 42 and from there via further lines 43 und 44 and accordingly assigned branch lines.

The parameters measured by the sensors (not shown) are transmitted by the monitoring system F via a line 35 to the computer-controlled monitoring and control system G for further processing. As already explained above in the FIGS. 1 to 3A, the cell culture chamber 20 is equipped with at least one channel 4 for sensor connection. The sensors and the relevant assigned channel 4 of the membrane plate 1 are adjusted to each other in such a way so as to ensure sterility.

In summary, the closed cell culture system 30 equipped with cell culture chambers 20 according to the invention is able to simulate highly complex biological processes in real time and under almost in-vivo conditions, i.e. as in living organisms.

1. Cell culture chamber (20) for a closed cell culture system serving to continuously supply different cells with liquid nutrient media, growth factors, gases and the like, characterized by the fact that the cell culture chamber (20) is materially composed of the following components:
a) a membrane plate (1) with a membrane (2) serving to accommodate at least one cell culture and equipped with a number of channels (4,4′, 4″, 4′″) for supplying liquid, gassing and sensor connection;
b) a transparent glass pane (3) which is placed on one side of the membrane plate (1) while provided for observing the inside of the cell culture chamber (20) from the side mentioned above; and
c) a cover plate (5) which is placed on the opposite side of the membrane plate (1) and which is provided with an incorporated, transparent glass pane (6) for illuminating the inside of the cell culture chamber (20) from this opposite side with the aid of an assigned lighting system.

2. Cell culture chamber according to claim 1, characterized by the fact that the transparent glass pane (3) is fixed on the membrane plate (1) in the area of the underside of the membrane plate (1) for the purpose of observing the inside of the cell culture chamber (20).

3. Cell culture chamber according to claim 1, characterized by the fact that the cover plate (5) forms a cell culture chamber cap with a securely integrated transparent glass pane (6), such cell culture chamber cap being fixed in a releasable manner on the upper side of the membrane plate (1).

4. Cell culture chamber according to claim 1, characterized by the fact that the cell culture chamber cap (5) as well as the underside of the membrane plate (1) show an opening for the accommodation and fixation of the respective glass pane (6 resp. 3).

5. Cell culture chamber according to claim 1, characterized by the fact that the glass pane (6, 3) is a sapphire glass pane.

6. Cell culture chamber according to claim 1, characterized by the fact that, for the fixation of the membrane (2) on the membrane plate (1), a retaining ring (7) is provided, which can, with the aid of the cell culture chamber cap (5), be pressed on the periphery of the membrane (2) so that the latter can be fixed.

7. Cell culture chamber according to claim 1, characterized by the fact that, on the side of the cell culture chamber cap (5) facing the membrane plate (1), a joint ring (8) is provided, by which the cell culture prepared on the membrane (2) is aseptically closed when the cell culture chamber (20) is closed.

8. Cell culture chamber according to claim 1, characterized by the fact that a constant, continuous gassing is enabled by means of a suitable compartmentalization of the cell culture chamber (20) via the respectively assigned channels (4′, 4′″) with freely selectable concentrations of the most various gases.

9. Cell culture chamber according to claim 1, characterized by the fact that the membrane plate (1) can be fixed at its side opposite the cell culture chamber cap (5) on an assigned retainer plate (10) for integration in the cell culture system, this retaining plate (10) being equipped with an integrated heating for the cell culture chamber (20).

10. Cell culture chamber according to claim 9, characterized by the fact that the heating is an electrical heating.

11. Cell culture chamber according to claim 1, characterized by the fact that the membrane (2) is a gas-permeable biofoil.
